(19) United States
(12) Patent Application Publication
(10) Pub. No.: US 2006/0062841 A1
(43) Pub. Date: Mar. 23, 2006

(54) Liposomal vectors

(76) Inventors: Leaf Huang, Durham, NC (US); Feng Liu, Durham, NC (US)

Correspondence Address: BAKER & BOTTS, 30 ROCKEFELLER PLAZA, NEW YORK, NY, 10112, US

(21) Appl. No.: 11/219,507

(22) Filed: Sep. 1, 2005

Related U.S. Application Data
(60) Provisional application No. 60/606,275, filed on Sep. 1, 2004.
(60) Provisional application No. 60/609,314, filed on Sep. 13, 2004.

(51) Int. Cl. A61K 48/00; A61K 9/127; C12N 15/88; A61K 36/37

(52) U.S. Cl. 424/450; 435/458; 424/759; 514/44

(57) ABSTRACT

The present invention provides for liposomal vectors which inhibit inflammation and/or enhance expression of transferred genes. It is based, at least in part, of the discovery that vectors comprising a nucleic acid carrying a gene of interest and a cationic liposome containing an immunosuppressive agent induce lower levels of inflammatory cytokine relative to conventional lipolex vectors, as well as on the discovery that a component may be incorporated into the liposome which enhances expression of the gene of interest.

### CROSS-REFERENCE TO RELATED APPLICATIONS

This application claims priority to U.S. Provisional Application Nos. 60/606,275 and 60/609,314, filed Sep. 1, 2004 and Sep. 13, 2004, respectively, each of which are incorporated by reference in their entireties.

### STATEMENT REGARDING GOVERNMENT SUPPORT

This invention was made with government support under A148851 by the National Institutes of Health, so that the United States Government has certain rights herein.

### 1. INTRODUCTION

The present invention relates to a liposomal vector and a nucleic acid carrying a gene of interest, where the vector further comprises a suppressor of inflammation and/or a component that enhances expression of the gene of interest.

### 2. BACKGROUND OF THE INVENTION

Using nucleic acids as drugs for gene therapy purposes has led to the development of sophisticated and efficient DNA carriers, also called vectors. A diverse number of vectors, viral and nonviral, have been developed for gene therapy. Viral vectors, at present, dominate in clinical trials because they are highly efficient in transducing cells. However, viral vectors are immunogenic and potentially mutagenic, thus nonviral vectors have gained increased attention recently (1). Since the first report by Felgner et al. describing cationic lipid-based delivery systems (lipoplex), a significant number of families of cationic vectors have been synthesized (2). Nonviral vectors are particularly suitable with respect to simplicity of use, ease of large-scale production and lack of specific immune response (3). Among the nonviral vehicles, the lipoplexes (complexes of cationic liposome/pDNA) are the most studied and represent the most promising approaches for human clinical trials. They have been studied in several clinical trials for treatment of cystic fibrosis, cancer, and cardiovascular diseases (4).

However, the lipoplexes are not without overt toxicity. Dose-dependent pulmonary inflammation was reported after instillation of the lipoplexes into the lungs of mice (5). The inflammation was characterized by increased concentrations of the proinflammatory cytokines, including tumor necrosis factor α (TNF-α), interleukin 6 (IL-6), and interferon γ (IFN-γ) in the bronchoalveolar lavage fluid. Some side effects which may be related to inflammation were also observed in a clinical study in which cystic fibrosis (CF) patients were subjected to either aerosolized cationic liposomes alone or the lipoplexes (6). Each of the lipoplex-treated patients, but not the cationic liposome-treated controls, exhibited mild flu-like symptoms, including fever and myalgia over a period of approximately 24 h. The inflammatory response becomes severe in the animal model when the lipoplexes are systemically administrated. Intravenous injection of the lipoplexes induces a rapid induction of large quantities of proinflammatory cytokines in blood, such as TNF-α, IL-12, IL-6, and IFN-γ (7-10). Twenty-four hours after intravenous injection of the lipoplexes at doses required to produce therapeutically relevant levels of transgene expression, mice invariably appear scruffy and lethargic because of the cytokines induced (8). Mortality is frequently observed at the higher doses used. Although decreasing the dose can prevent this mortality, such a reduction also results in a disproportionate decrease in transgene expression, highlighting the narrow therapeutic index characteristic of these vectors (8). The cytokines, secreted after intravenous injection of the lipoplexes, is not only toxic but also cause short-term transgene expression and refractory behavior on repeated dosing at frequent intervals (9, 11).

Systemic injection of naked DNA does not result in cytokine induction even though it contains immunostimulatory CpG motifs. Several studies have demonstrated that the liver is the main organ to clear the naked plasmid DNA (25, 26). Naked DNA was predominantly taken up by the liver nonparenchymal cells, mainly by the liver endothelial cells (26). When DNA is complexed with positively charged liposomes to form lipoplexes (cationic liposome/DNA complexes), the pharmacokinetic behavior of the DNA is changed due to the fact that lipoplexes interact with various components in blood and exhibit a complicated distribution profile. Upon injection into blood, the lipoplex with a high charge ratio (+/−), for example 6/1, rapidly formed aggregates with serum proteins. The aggregates are efficiently entrapped in pulmonary vasculature, and deliver pDNA to the target cells (lung endothelial cells) because of a sufficient interaction of the vector with the cells. Some of lipoplexes, on the other hands, are guided by the binding proteins, opsonin proteins, to the immune cells (including lung macrophage and RES cells in the liver and spleen) (27). Thus, cationic liposomes play a synergistic role, delivering pDNA to target cells (for example, lung endothelial cells) and assisting the introduction of DNA into immune cells where CpG motif DNA stimulates the inflammatory response.

It appears that unmethylated CpG sequences in bacterial DNA or plasmid DNA (pDNA) are responsible for the immunostimulatory response, resulting in the induction of the cytokines (12). In bacterial DNA, CpG sequences are usually unmethylated, whereas in mammalian DNA about 75% of the CpG are methylated to 5′-methycytosine (13). This structural difference between bacterial and mammalian DNA is a signal for the induction of innate immunity to microbial infections. Therefore, the side effects of the lipoplexes, mainly inflammatory, must be well controlled when these vectors are used in gene therapy.

Prior to the present invention, an inflammatory response was essentially invariably associated with administration of gene transfer complexes (lipoplexes) composed of cationic lipids and plasmid DNA (pDNA). In addition to causing inflammation, the unmethylated CpG in pDNA has been shown to induce apoptosis of lung endothelia cells when delivered systemically (9).

Another concern is the potential for augmenting an antibody response to the transgene product or to the pDNA itself (21). The T helper-1 response to the immunostimulatory CpG motifs makes pDNA an excellent adjuvant for immunization to a variety of protein antigens (22, 23). This property is useful and indeed has been exploited for genetic vaccines, but is deleterious for the treatment of genetic disorders that require repeated delivery of lipoplexes. Bacterial DNA also has been shown to be capable of inducing the production of anti-DNA antibodies in mice (24). This finding suggests the possibility of developing an autoimmune response in patients as a result of treatment (21). There was therefore a need to develop vectors which could efficiently effect gene transfer but not provoke as great an inflammatory response as conventional lipoplex vectors.

### 3. SUMMARY OF THE INVENTION

The present invention provides for liposomal vectors which inhibit inflammation and/or enhance expression of transferred genes. It is based, at least in part, of the discovery that vectors comprising a nucleic acid carrying a gene of interest and a cationic liposome containing an immunosuppressive agent induce lower levels of inflammatory cytokine relative to conventional lipoplex vectors, as well as on the discovery that a component may be incorporated into the liposome which enhances expression of the gene of interest.

In particular embodiments, the present invention provides for a non-viral vector comprising a nucleic acid carrying a gene of interest, in expressible form, and a cationic liposome containing one or more suppressor of inflammation and/or one or more component that enhances expression of the gene of interest. In non-limiting embodiments, the component that enhances expression of the gene of interest is a ligand of a transcriptional activator which is capable of binding to a response element in the vector-borne nucleic acid.

In further embodiments, the present invention provides for methods of delivering a gene of interest to a cell comprising contacting the cell with a liposomal vector as described above. Vectors which comprise an enhancer of gene expression may be used to effect more productive gene transfer. Where the cell exists in an environment that includes cells of the immune system, vectors of the invention which comprise a suppressor of inflammation may induce less inflammation relative to conventional lipoplex vectors. Moreover, vectors which comprise an enhancer of gene expression, because they are more potent than conventional vectors and may be used at lower concentrations, may further provide for a lower level of inflammatory response.

### 4. BRIEF DESCRIPTION OF THE FIGURES

FIG. 1. Schematic depiction summarizing the function of inflammatory suppressors which interrupt the cytokine production by inhibiting NF-κB at one or multiple activation steps of the signaling pathway. This graphic representation is modified from reference 16.

FIG. 2A-B. Dynamic change in the turbidity of DOTAP liposomes, DEX/DOTAP liposomes and DEX suspension (A). In vivo gene transfer with the lipoplexes and safeplexes (B). The absorbance (a) at 600 nm was measured at different times. Luciferase activity (b) in mouse organs was detected 6 h after the injection of lipoplexes or safeplexes containing 25 μg luciferase plasmid DNA. The data are expressed in RLU (relative light units) per mg of total protein extracted the tissue as mean ± S.D. (n=3). P>0.5

FIG. 3A-C. Comparison the levels of TNF-α in blood (a), organs (b) and the blood levels of IL-12 and IFN-γ (c) after injection of the lipoplexes and safeplexes. The charge ratio of DOTA to DNA (+/−) (a) was varied from 0 to 12 and (b) and (c) was fixed at 6 to 1. The levels of cytokines were detected 2 h (a,b) or 6 h (c) after injection of the lipoplexes and safeplexes containing 25 μg pDNA. P<0.001 (three mice in each group).

FIG. 4A-B. Effect of (A) DEX dose and (B) time course on suppressing TNF-α. Mice were (three in each group) injected with the lipoplexes or safeplexes with (A) a varied ratio of DEX to DOTAP and (B) with a ratio of DEX to DOTAP of 1/10 (w/w). (a, b) The charge ratio of DOTAP to DNA was fixed at 6 to 1. P>0.5, P<0.001

FIG. 5. Inhibition of TNF-α production by other suppressors. The levels of TNF-α in the blood were analyzed 2 h after the injection of lipoplexes and safeplexes with a charge ratio of DNA to DOTAP of 1/6 (mol/mol). P<0.001 (n=3).

FIG. 6. Schematic description of blocking the signaling pathways of NF-kappa B and AP-1 by using Capsaicin and SB 203580.

FIG. 7A-B. (A) Gene expression in mouse lung after luciferase gene transferred with the safeplexes. (B) Inhibition of TNF-alpha using the safeplexes, of DOTAP/capsaicin or DOTAP/SB 203560.

FIG. 8. The signaling pathway of the nuclear receptor FXR.

FIG. 9. Enhancement of gene expression by activation of FXR signaling pathway using DOTAP/CDCA liposomes.

### 5. DETAILED DESCRIPTION OF THE INVENTION

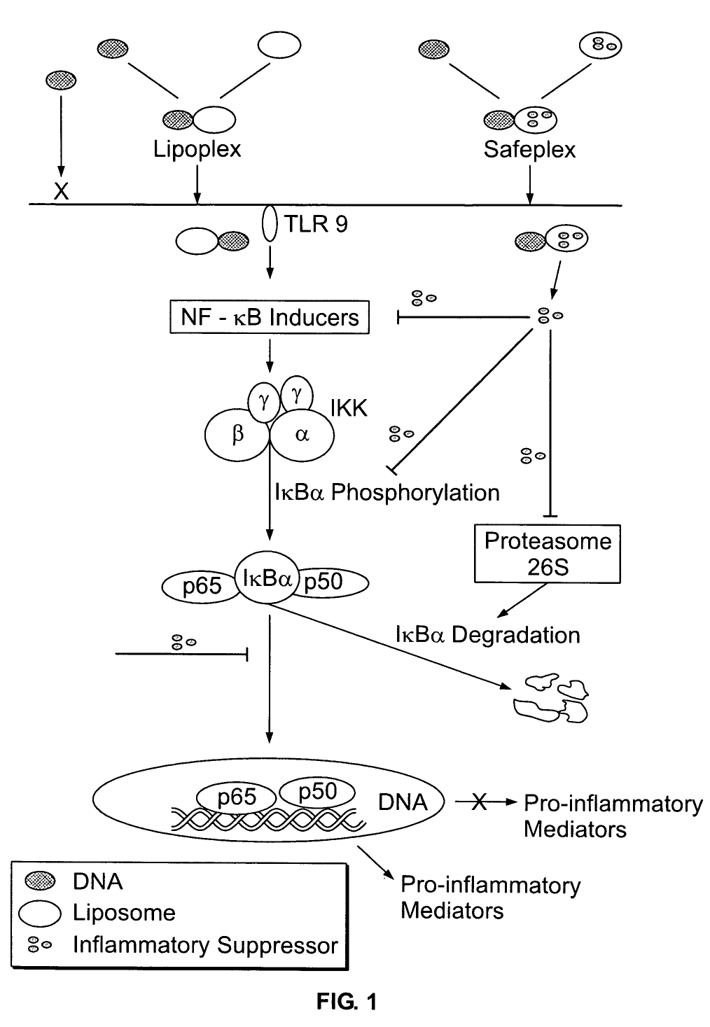

The present invention relates to a liposomal, preferably non-viral gene transfer vector that is engineered to increase its safety profile and therapeutic index. A rationale for designing this vector, as shown as a non-limiting embodiment in FIG. 1, is encapsulation of one or more inflammation suppressors into a cationic liposome to form a new type of complex, i.e., a cationic liposome/inflammation suppressor/nucleic acid molecule. In other non-limiting embodiments, the present invention provides for a cationic liposome/nucleic acid complex wherein the liposome additionally or alternatively comprises one or more component that enhances expression of a gene of interest comprised in the nucleic acid; in specific embodiments, the component is a transcriptional enhancer protein or a transcriptional enhancer complex.

The nucleic acid molecule, inflammation suppressor and/or enhancer component may be delivered together into individual target cells. Where the target cell would otherwise increase expression of one or more cytokine in response to the liposome/nucleic acid complex, upon release in the cell cytoplasm of the inflammatory suppressor(s) from the vector, cytokine production is inhibited by, for example, inhibiting the NF-κB or AP-1 inflammatory pathway at one or multiple activation steps. The liposomal vectors of the invention, referred to as “safeplex” vectors can be used for the systemic delivery of proteins and expressible nucleic acid molecules such as genes. The safeplex vector has been successfully used to intracellulary deliver many inflammatory suppressors such as glucocorticoids, non-steroidal anti-inflammatory drugs (NSAIDs), NF-κB inhibitors such as capsaicin, AP-1 inhibitors such as SB 203580, and natural compounds from herbal medicines. Moreover, the safeplex vector has been shown to dramatically decrease proinflammatory cytokines induced by plasmid DNA. In other specific non-limiting embodiments of the invention, expression of a gene of interest carried in a safeplex vector has been enhanced by incorporating, into the liposome, an expression enhancer; for example, the safeplex vector has successfully been used to intracellularly deliver proteins such as FXR and CDCA to enhance nuclear nucleic acid transcription.

For purposes of clarity, and not by way of limitation, the detailed description of the invention is divided into the following subsections:

(i) liposome/nucleic acid vectors;

(ii) vectors comprising liposomes containing suppressors of inflammation;

(iii) vectors comprising liposomes containing enhancers of gene expression; and

(iv) uses of the present invention.

### 5.1 Liposome/Nucleic Acid Vectors

The vectors of the invention comprise liposome/nucleic acid complexes. In preferred embodiments, the liposome is physically associated with the nucleic acid by attraction between opposite molecular charges; as DNA tends to be negatively charged, in preferred embodiments the liposome is positively charged, i.e., a cationic liposome. The cationic liposome may be formed, for example, from materials known in the art, such as, but not limited to, 1,3-dioleoyl-3-trimethylammonium-propane, N,N-dioleyl-N,N-dimethylammonium chloride (DODAC), 3-beta-(N-(N′,N′-dimethylaminoethane) carbamoyl) cholesterol (DC-Chol), N-(1-(2,3-dioleyloxy)propyl)-N,N,N-trimethylammoniumchloride (DOTMA), 1,3-dioleoyl-3-trimethylammonium-propane (DOTAP), N-(1,2-dimyristyloxyprop-3-yl)-N,N-dimethyl-N-hydroxyethyl ammonium bromide (DMRIE), DORIE, N-(1-(2,3-dioleyloxy)propyl)-N-(2-(sperminecarboxamido)ethyl)-N,N-dimethyl ammonium trifluoro-acetate (DOSPA), GAP-DLRIE, DMDHP, or diheptadecylamidoglycyl spermidine (DOGS). See, e.g., Raghavan and Chaudhuri, 1998, Drug Delivery Technology 392:25-30. The liposome may be formed using standard techniques.

The nucleic acid molecule comprised in a vector according to the invention comprises a gene of interest. Virtually any nucleic acid sequence may serve as a gene of interest, depending on its intended purpose. In particular, non-limiting embodiments, the gene of interest may encode a nucleic acid or a protein which has a reporter, therapeutic, diagnostic, or industrial function. The gene of interest is preferably in expressible form, meaning that it is operably linked to an element that promotes its expression. Suitable elements include promoter and enhancer sequences, sequences that promote translation, sequences that promote entry into the nucleus, and other expression regulatory elements (including but not limited to activator, supressor, and silencer sequences). In specific non-limiting embodiments described below, the gene of interest may be operably linked to an element which binds to a transcriptional activator or a transcriptional repressor molecule. Preferably, the nucleic acid sequence is not infectious. Preferably, the nucleic acid sequence is not a virus. In a particular non-limiting embodiment, the nucleic acid molecule is a plasmid. The nucleic acid may be DNA or RNA, may be antisense RNA, siRNA, catalytic DNA, catalytic RNA, etc. In specific, non-limiting embodiments of the invention, a nucleic acid containing a gene of interest may be modified to decrease the number of unmethylated CpG motifs.

In particular non-limiting embodiments, the non-viral vector has a liposome/nucleic acid charge ratio (“L:N charge ratio”) of cationic to negative charge ((+) liposomal charge: (−) nucleic acid charge) of about 0.5:1, 1:1, 2:1, 5:1, 10:1, 15:1, 20:1, 40: 1, or 100:1, and/or between 0.5:1 and 100:1, between 0.5:1 and 40:1, between 0.5:1 and 20:1, between 0.5:1 and 10:1, between 2:1 and 40:1, between 2:1 and 20:1, or between 2:1 and 15:1. In a specific non-limiting embodiment, the L:N charge ratio is about 6:1. In a specific non-limiting embodiment, the L:N charge ratio is 12:1.

### 5.2 Vectors Comprising Liposomes Containing Suppressors of Inflammation

In preferred non-limiting embodiments, the present invention delivers a gene of interest to target cells and an inflammation suppressor (also referred to as an “inflammatory suppressor”) to cells of the immune system, although the present invention further encompasses the use of agents that inhibit cytokine production in the target cell which may otherwise trigger an immune cell effected inflammatory response.

The inflammation suppressor and pDNA can be co-delivered into the same type of cells, including target cells and cells of the immune system. When both are delivered into the lung endothelial target cells, for example, the inflammation suppressor does not impede gene transfection. However, when they are co-delivered into the immune cells, as shown in FIG. 1, the released inflammatory suppressor will find its own target to inhibit cytokine production. The anti-inflammatory effect by the inflammation suppressors can be achieved at very low dose because the suppressors carried by the safeplex vector are efficiently delivered to the immune cells. In contrast, when inflammation suppressors are administrated in other formulations through intravenous or intraperitoneal injection, a higher dose is needed, which may induce toxicity.

In particular non-limiting embodiments, the present invention relates to vector comprising a cationic liposome, one or more inflammation suppressor, and a nucleic acid molecule comprising a gene of interest in expressible form. In a non-limiting embodiment, the inflammation suppressor is encapsulated in the cationic lipid. In another non-limiting embodiment, the vector further comprises a pharmaceutically acceptable carrier. Preferably, the vector produces an immune response and/or cytokine production which is reduced, relative to a comparable control vector lacking an inflammatory repressor, by at least about 30 percent, 40 percent, 50 percent, 60 percent, 70 percent, 80 percent or 90 percent.

An inflammation suppressor, in the context of the invention, means any agent that reduces or inhibits an inflammatory response which would otherwise be induced by the liposomal vector. The inflammatory suppressor may act at any stage of the inflammatory process, including but not limited to recognition and reaction to unmethylated CpG sequences, induction of cytokine, recruitment of immune cells, or expression of immune effector functions.

In one set of non-limiting embodiments of the invention, the inflammation suppressor may be a glucocorticoid such as betamethasone, budesonide, cortisone, dexamethasone, hydrocortisone, methylprednisolone, prednisolone, prednisone, triamcinolone, or any combination thereof.

In another set of non-limiting embodiments of the invention, the inflammation suppressor may be a nonsteroidal anti-inflammatory drug such as indomethacin, ibuprofen, naproxen, aspirin, acetaminophen, or an agent that inhibits cyclooxygenase such as nimesulide, or any combination thereof.

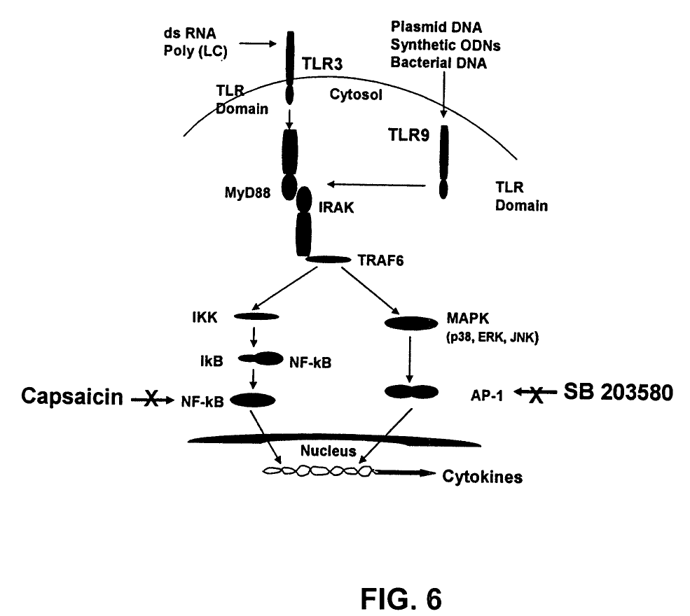

Toll-like receptor 9 (TLR9) has been identified as the receptor involved in the recognition of immunostimulatory CpG motifs to activate NF-κB and AP-1 (30, 31). In another set of non-limiting embodiments of the invention, the inflammation suppressor functions to inhibit NF-κB or AP-1 at one or multiple activation steps of the signaling pathway (in FIG. 1 and FIG. 6). For example, but not by way of limitation, the inflammation suppressor may be an inhibitor of NF-kappa B such as gliotoxin or an alkaloid such as capsaicin or other capsaicin-like analogs such as the vanillylamides and the nonivamides. Alternatively, the inflammation suppressor may be an inhibitor of AP-1 such as SB 203580.

In another set of non-limiting embodiments of the invention, the inflammation suppressor may be a natural compound such as tetrandrine, extract of Periwinkle, extract of Tripterygium, or any combination thereof.

In another set of non-limiting embodiments, the inflammation suppressor is a specific inhibitors of the CpG signaling pathway such as chloroquine and quinacrine (10).

In another set of non-limiting embodiments of the invention, the inflammation suppressor may be an antisense, siRNA, or catalytic DNA or RNA, that inhibits expression of a cytokine such as IL-1 alpha, IL-1 beta, IL-2, IL-3, IL-4, IL-5, IL-6, IL-7, IL-8, IL-10, IL-11, IL-12, IL-13, IL-15, IL-16, IL-17, IL-18, IL-19, IL-20, IL-22, IL-29), leukemia inhibitory factor (LIF), oncostatin M, ciliary neurotrophic factor (CNTF), cholinergic differentiation factor (CDF), interferons (e.g., IFN-alpha, IFN-beta, IFN-gamma), Granulocyte-macrophage colony-stimulating factor (GM-CSF), Interleukin-3 (IL-3), fibroblast growth factor (FGF), tumor necrosis factor (e.g., TNF-alpha, TNF-beta), transforming growth factor (e.g., TGF-alpha, TGF-beta), gonadotropin, nerve growth factor (NGF), platelet-derived growth factor (PDGF), macrophage inflammatory protein 1 (e.g., MIP-1 alpha, MIP-1 beta), and melanoma growth stimulating activity (MGSA).

In other embodiments, inflammation suppressors identified using assays as set forth below may be incorporated into vectors of the invention.

In still further embodiments, more than one inflammation suppressor agent may be comprised in a vector according to the invention. Specific non-limiting embodiments include combinations of steroidal and non-steroidal anti-inflammatory agents, a NF-κB inhibitor and a non-steroidal agent, or a mixture of an extract of Tripterygium, prednisone, an inhibitor of NF-kappa B, and an agent that inhibits cyclooxygenase.

In particular non-limiting embodiments, the inflammation suppressor is at a dose of approximately 1, 2, 3, 4, 5, 6, 7, 10, 12, 30, 60, or 100 micrograms. In specific non-limiting embodiments, the dose of the inflammatory suppressor is 3.2, 8, 16, 32, or 64 micrograms.

In particular non-limiting embodiments, the weight/weight ratio of inflammatory suppressor to cationic lipid (“ISw:LDw”) used to produce the inflammatory suppressor-containing liposomes comprised in the vectors of the invention is about 1:1, 1:2.5, 1:5, 1:7.5, 1:10, 1:15, 1:25, or 1:50, and/or between 1:1 and 1:50, between 1:2.5 and 1:20, or between 1:2.5 and 1:10. In a specific non-limiting embodiment, the ISw:LDw ratio is 1:10. In another specific non-limiting embodiment, the molar ratio of inflammatory suppressor to cationic lipid is 1:6.

Liposomes containing inflammation suppressor may be prepared from solutions containing lipid and inflammatory suppressor using methods known in the art.

In one specific, non-limiting embodiment, the inflammation suppressor comprises dexamethasone and the cationic lipid comprises DOTAP.

### 5.3 Vectors Comprising Liposomes Containing Enhancers of Gene Expression

The present invention further provides for liposomal gene transfer vectors comprising a liposome, a nucleic acid molecule comprising a gene of interest, in expressible form, and one or more component that enhances expression of the gene of interest. Such vectors may optionally further comprise one or more inflammation suppressor as set forth above.

A component that enhances expression of the gene of interest is a component that enhances uptake (including nuclear uptake), transcription, or translation of the gene of interest. The component may be a peptide or protein, a nucleic acid, or other species of molecule.

In preferred, non-limiting embodiments of the invention, the component is an agent that directly or indirectly binds to an expression control sequence that enhances transcription of the gene of interest. For example, the component may be a transcriptional activator that binds to an expression control sequence operably linked to the gene of interest comprised in the nucleic acid borne by the vector (or a nucleic acid encoding said activator, in expressible form). Alternatively, the component may be a ligand for such a transcriptional activator that promotes binding, or a ligand for a transcriptional suppressor which releases binding (thereby promoting transcription) to a nucleic acid response element. In various embodiments, one or more component may be comprised in the liposomal vector of the invention. In a non-limiting embodiment, the transcription enhancing proteins are encapsulated in a cationic liposome comprised in a vector of the invention.

In one specific, non-limiting example, a liposomal vector of the invention may comprise a farnesoid x receptor (FXR) and/or its ligand chenodeoxycholic acid (CDCA), wherein the nucleic acid of the vector comprises a gene of interest operably linked to a farsenoid x response element (FXRRE). In related non-limiting embodiments, similar expression enhancing components may be incorporated based on comparable molecules of the nuclear receptor superfamily which includes, in addition to FXR, CAR (constitutive androstane receptor), PXR (pregnane x receptor), LXR (liver x receptor), PPAR (peroxisome proliferator-activated receptor), and their ligands, such as CDCA (chenodeoxycholic acid). In such embodiments, FXR (or analogous molecule) may be provided via a second gene of interest encoding FXR in expressible form which can be either comprised in the same nucleic acid as the first gene of interest or as a separate nucleic acid comprised in the liposome, or may be provided as a protein within the liposome. Of note, where FXR/FXRRE is used to enhance transcription of the gene of interest, if the target cell expresses sufficient levels of endogenous FXR, vectors may be used which contain ligand (CDCA) which promotes FXR/FXRE binding, without a source of FXR.

In alternative specific non-limiting embodiments, a tetracycline-based system may be used to selectively enhance transcription of the gene of interest. According to such embodiments, a tetracycline response element (the Tet operator) is operably linked to the gene of interest, and the mutant Tet-On repressor protein is supplied, either via a nucleic acid encoding it or in protein form (33). After the liposomal vector transfers the gene of interest to a target cell, administering an effective amount of tetracycline to the cell may be used to induce transcription of the gene of interest.

In non-limiting embodiments, where an enhancer component is supplied as a nucleic acid, such as a nucleic acid encoding a component which is a protein, the amount present in the liposome may be in the same ratios set forth in section 5.1. In other non-limiting embodiments, where the component is a protein or other molecule, the amount present relative to the amount of lipid may be in ratios set forth for inflammation suppressors in section 5.2.

### 5.4 Uses of the Present Invention

The present invention provides for methods for delivering, to a cell, a nucleic acid molecule comprising a gene of interest, in expressible form, and an inflammation suppressor and/or expression enhancer. The method comprises contacting the cell with an effective amount of a liposomal vector, as described above. Preferably, where the cell is in an environment that comprises cells of the immune system, the vector is substantially non-immunostimulatory. By this method, the immune response of the cell to the nucleic acid molecule of the vector may be prevented, inhibited or reduced by the presence of the inflammation suppressor. Accordingly, the cell may respond to its exposure to the vector by limiting or reducing its production of one or more cytokines.

In certain non-limiting embodiments, the present invention relates to a method for treating a subject, the method comprising administering to the subject in need of such treatment an effective amount of a liposomal vector, as described above. The immune response of the subject to the non-viral vector may be determined by any means known to one of ordinary skill in the art (e.g., a physician), and may include measuring the level of a cytokine or other index of inflammation to determine the level of inflammation, if any, induced. The non-viral vector of the invention may be administered to the subject by any means known to one of ordinary skill in the art, such as but not limited to administration by intravenous, intraperitoneal, intrathecal, subcutaneous, intramuscular, topical, oral, rectal, transmucosal, intravaginal, intraocular, intranasal, intramedullary, intracentricular or inhalation route. Where administration is to be to a specific site, such as a wound, a surgical site, a stent implantation site, or a tumor, in non-limiting embodiments the liposomal vectors of the invention may be locally applied, for example by instillation or injection. In a non-limiting embodiment, the vector is formulated for systemic administration. Techniques for formulation and administration may be found, for example, in “Remington's Pharmaceutical Sciences,” Mack Publishing Co., Easton, Pa. For intravenous injection, the vector may be formulated in an aqueous solution, preferably in physiologically compatible buffers such as for example Hanks' solution, Ringer's solution, or physiological saline buffer. For transmucosal administration, penetrants appropriate to the barrier to be permeated are used in the formulation. Such penetrants are generally known in the art.

For therapeutic uses, the liposomal vectors of the invention may be administered in combination with other agents. For example, but not by way of limitation, the vectors may be administered together with an amount of an anti-inflammatory agent or immune suppressant. Where the liposomal vector of the invention comprises an inflammation suppressor, the amount of anti-inflammatory agent or immune suppressant which is co-administered may be reduced relative to the amount which would be administered if the vector lacked the inflammation suppressor.

The present invention also relates to screening assays for identifying anti-inflammatory agents. The screening assay is performed by contacting a population of cells with a non-viral vector having a cationic liposome, a nucleic acid molecule comprising a gene of interest (such as a reporter gene) in expressible form, and the candidate anti-inflammatory agent, wherein the vector is substantially non-immunostimulatory. Preferably the population of cells comprises cells which are immune cells (e.g., B cells and/or T cells and/or antigen presenting cells). After contacting the vector, the immune response of the cell to the vector is measured relative to a control vector, wherein a reduced immune response (as compared to control) indicates that the agent has anti-inflammatory properties. The control vector may be any suitable control vector such as the vector having the cationic liposome and the nucleic acid molecule, but not the candidate agent. Candidate agents may be obtained from laboratory chemical syntheses, and thus may be non-naturally occurring. Alternatively, candidate agents may be substantially purified from a natural source, such as by obtaining an extract from a medicinal herb. Non-limiting examples of natural sources include a protozoan, a plant, and a fungus and specific medicinal plants such as, but not limited to, a species of Achillea, Artemisia, Crataegus, Digitalis, Echinacea, Glycyrrhiza, Hypericum, Marrubium, Matricaria, Nepeta, Passiflora, Plantago, Symphytum, Tanacetum (Chrysanthemum), Taraxacum, Tilia, and Valeriana. The screening assays of the invention may be performed in vitro or in vivo.

In the therapeutic or the assay context, the efficacy of the vectors of the invention in avoiding inflammation may be evaluated by measuring the level of one or more cytokine produced. For example, the method may reduce the production of a cytokine by at least 50%, 60%, 70%, 80%, 90%, 95%, or 99% relative to control. Any relevant cytokine may be measured such as, but not limited to, growth hormone (somatotropin), prolactin, granulocyte-macrophage colony stimulating factor (GM-CSF), myelomonocytic growth factor, interleukins (e.g., IL-1 alpha, IL-1 beta, IL-2, IL-3, IL-4, IL-5, IL-6, IL-7, IL-8, IL-10, IL-11, IL-12, IL-13, IL-15, IL-16, IL-17, IL-18, IL-19, IL-20, IL-22, IL-29), leukemia inhibitory factor (LIF), oncostatin M, ciliary neurotrophic factor (CNTF), cholinergic differentiation factor (CDF), interferons (e.g., IFN-alpha, IFN-beta, IFN-gamma), Granulocyte-macrophage colony-stimulating factor (GM-CSF), Interleukin-3 (IL-3), fibroblast growth factor (FGF), tumor necrosis factor (e.g., TNF-alpha, TNF-beta), transforming growth factor (e.g., TGF-alpha, TGF-beta), gonadotropin, nerve growth factor (NGF), platelet-derived growth factor (PDGF), macrophage inflammatory protein 1 (e.g., MIP-1 alpha, MIP-1 beta), and melanoma growth stimulating activity (MGSA).

Accordingly, in specific embodiments, the vectors of the invention represent a new generation of non-viral vector for gene therapy and offer the advantages of 1) efficiently delivering a gene of interest to target cells, 2) inhibiting consequent cytokine production, and 3) enhancing nucleic acid nuclear transcription.

The present invention also provides a pharmaceutical kit comprising an effective amount of one or more non-viral vectors of the invention. In a non-limiting embodiment, the kit comprises, in at least one container, an effective amount of a safeplex vector.

The present invention will be better understood by the following exemplary teachings. The examples set forth herein do not and are not intended to limit in any manner the present invention.

### EXAMPLE 1

### Preparation of Lipoplexes and Safeplexes

All inflammatory suppressors were dissolved in organic solvents with concentration of 10 mg/ml (dexamethasone in methyl alcohol, prednisone in methyl alcohol and chloroform (1:1), indomethacin, tetrandrine and gliotoxin in chloroform). DOTAP in chloroform was added with (for the preparation of the safeplexes) or without (for the preparation of the lipoplexes) an inflammatory suppressor, and then was placed under a stream of nitrogen to evaporate the solvent until a thin lipid film formed at the bottom of a glass tube. It was further vacuum-desiccated for 1 h and then hydrated in 5% of dextrose solution to a final concentration of 10 mg DOTAP/ml (4 mg DOTAP/ml for tetrandrine and gliotoxin liposomes). The lipid suspension was briefly sonicated and then sequentially extruded through polycarbonate membrane of pore size of 0.2 μm. The DOTAP liposomes, suppressor/DOTAP liposomes, and pNGVL-3 luciferase plasmid DNA were separately diluted with 5% of dextrose solution to equal volumes. The lipoplexes or safeplexes were formed after adding DNA solution dropwise into the freshly prepared DOTAP or suppressor/DOTAP liposome solution in a glass tube while the tube was gently swirled. The resulting lipoplexes and safeplexes are translucent. The dilution was calculated such that a final volume of 200 μl of the lipoplexes or safeplexes was to be injected into one animal.

### EXAMPLE 2

### Evaluation of In Vivo Transgene Expression

The luciferase plasmid was used as a reporter gene. CD1 female (18-20 g) mice were injected intravenously with the lipoplexes or safeplexes with a charge ratio of 12 to 1 (+/−) and sacrificed 6 h after the injection. Each lung was collected and placed in 1 ml of ice-cold lysis buffer and homogenized with a tissue tearor (BioSpec Products, Bartlesville, Okla.) for 20 s at the highest speed. The homogenates were then centrifuged at 14,000 g for 5 min at 4° C. Ten microliters of the supernatant was analyzed with the luciferase assay system (Promega, Madison, Wis.) using an automated LB953 luminometer (Berthod Bad Wildbad, Germany). The protein content of the supernatant was measured with the Bio-Rad Protein Assay System (Bio-Rad, Hercules, Calif.). Luciferase activity was expressed as relative light units per milligram protein (RLU/mg protein).

### EXAMPLE 3

### Cytokine Assay in the Blood and Organs

At indicated time points (2 h for TNF-α, 6 h for IL-12 and IFN-γ) following the injection of a tested sample containing 25 μg of pDNA, the blood and organs (the liver, lung and spleen) were collected. The blood was allowed to clot on ice for at least 4 h and then centrifuged at 3000 g for 20 min at 4° C., and serum was collected for the cytokine assay. For the organ assay, 50 μg of each organ was added with 0.5 ml PBS buffer and homogenized for 20 s at the highest speed. The homogenates were frozen (in liquid nitrogen) and thawed (in 37° C. water bath) three times and then centrifuged at 14,000 g for 5 min. The supernatants were collected for the cytokine assay. The concentrations of cytokines (TNF-α, IL-12 and IFN-γ) were determined with mouse cytokine immunoassay kits (R&D System, Minneapolis, Minn.). Data were analyzed by the paired Student's t test using Excel. Data were considered statistically significant if P<0.01.

### EXAMPLE 4

### Physical Properties of the Safeplex Vector and its Gene Transfer

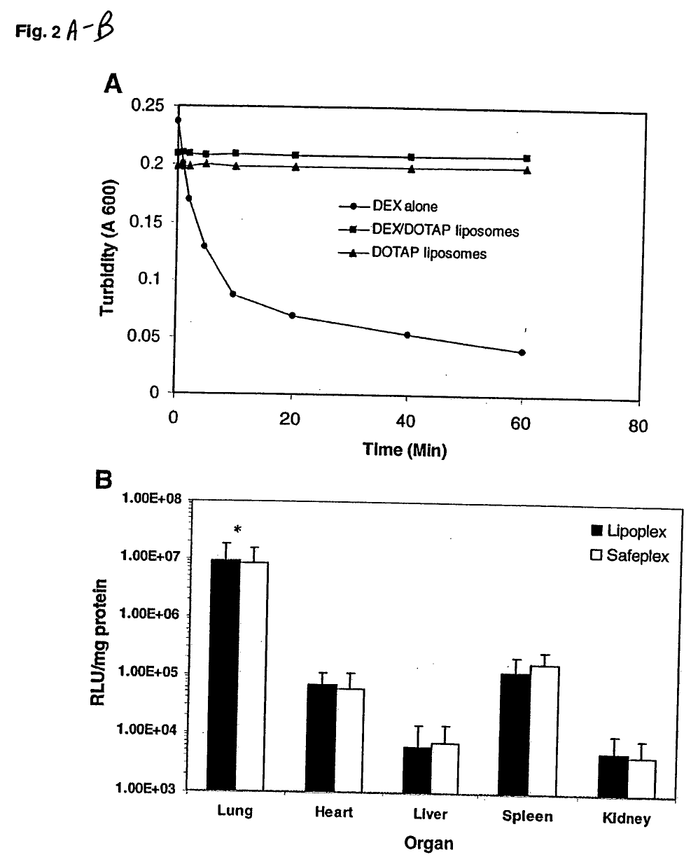

The procedure was performed to ensure that the encapsulation of an inflammatory suppressor into cationic liposomes would not change the physical properties of the liposomes such as the size and Zeta potential. DOTAP (1,3-Dioleoyl-3-trimethylammonium-propane) liposome has been extensively used as a nonviral vector in in vitro and in vivo gene transfer. Therefore, DOTAP was used as a control in this experiment. The data in Table 1 confirmed that when dexamethasone (DEX) was used as an inflammatory suppressor and encapsulated in DOTAP liposomes at a ratio of 1/10 (DEX/DOTAP, w/w), the size and Zeta potential of DOTAP/DEX liposomes did not change compared with the control. When DOTAP or DOTAP/DEX liposomes were mixed with plasmid DNA, the size for both particles was increased, indicating the formation of the lipoplexes (DOTAP/DNA complexes) or the safeplexes (DOTAP/DEX/DNA complexes). The safeplex, with a charge ratio of 6/1 (+/−), showed the similar size and Zeta potential as that of DOTAP liposomes. The DEX alone (without DOTAP) could not be formulated into a homogeneous solution following the same protocol for the liposome preparation. The rapid decrease in turbidity of this heterogeneous solution, as shown in FIG. 2a, indicates that the drug precipitated out of the solution. However, when DEX/DOTAP liposomes were formulated, the turbidity of the liposome solution did not change, which indicated that they formed a homogeneous solution and most of DEX was encapsulated by liposomes. Next we compared the efficiency of in vivo transfer gene using the lipoplexes and safeplexes. The data of in vivo gene transfer in FIG. 2b confirmed that the lipoplexes exhibited the same gene transfer pattern as the lipoplexes. There was no significant difference (p>0.5) in luciferase expression in the lung, which is the major organ expressing the transgene after intravenous injection of the lipoplexes. The above data clearly demonstrated that the suppressor encapsulated into the liposomes did not change the physical property of the gene vector and did not interfere with the gene transcription and translation in the transfected cells.

### EXAMPLE 5

### Inhibition of Cytokines by Safeplex

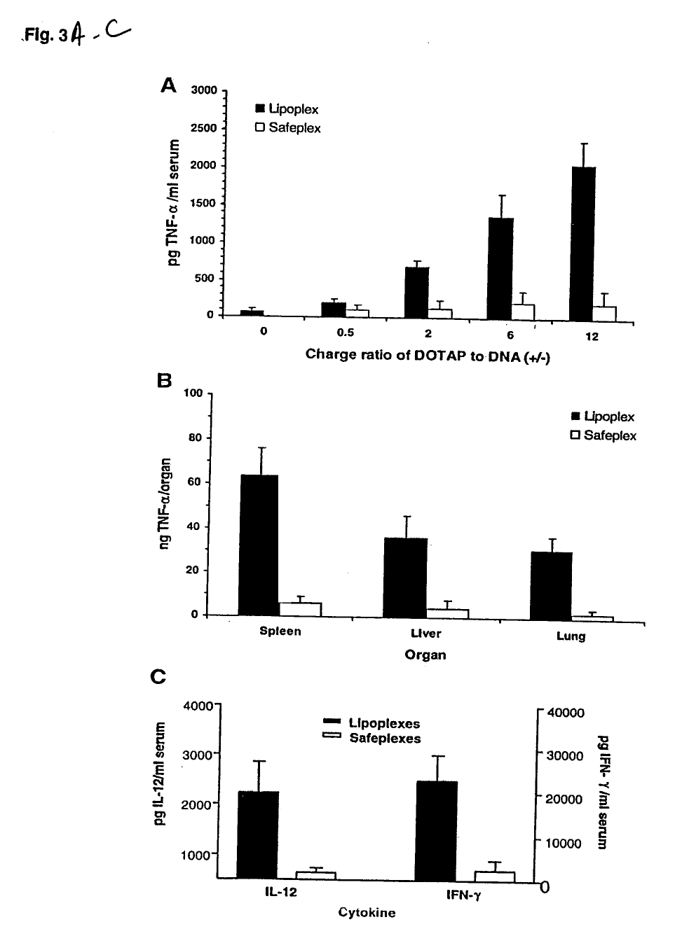

Tumor necrosis factor a (TNF-α) is one of the major cytokines induced by CpG motif DNA. It has been shown that intraperitoneal injection of DEX suppressed cationic lipid vector-mediated TNF-α induction (11). Therefore, TNF-α was used as an index and DEX was selected as the suppressor for evaluating effect of the safeplex on inhibiting cytokines. It is known that high levels of gene transfer into the lung requires a high charge ratio of cationic lipid to DNA, the higher charge ratio the higher efficiency of gene transfer. However, the data in FIG. 3a revealed that an increase in the charge ratio resulted in increased TNF-α production. The data showed that injection of plasmid DNA alone did not induce TNF-α, and a low level of TNF-αwas observed after injection of either the lipoplexes or safeplexes with a low charge ratio (+/−=0.5). The levels of TNF-α in blood increased as high as 2000 pg/ml when the charge ratio of the lipoplexes was increased to 12 to 1. However, when plasmid DNA was delivered by the safeplexes, the levels of TNF-α were significantly decreased, even at the highest charge ratio of 12 to 1 (P<0.01). It is known that the major organs involved in the production of cytokines are the spleen, followed by the liver and lung 14). Theoretically, the inflammatory suppressors delivered by the safeplex should accomplish their desired functions in these organs. The data in FIG. 3b confirmed that the levels of TNF-α in these organs were greatly decreased by the safeplex. In addition, the levels of other cytokines, IL-12 and IFN-γ, also decreased significantly at the ratio of 2/10 (more than 70% reduction of IL-12 and IFN-γ) (FIG. 3c). These data indicate that the safeplex can serve as a non-immunostimulatory gene vector to inhibit the inflammatory toxicity induced by CpG motif pDNA.

### EXAMPLE 6

### Effect of DEX Dose and Time Course on Suppressing TNF-α

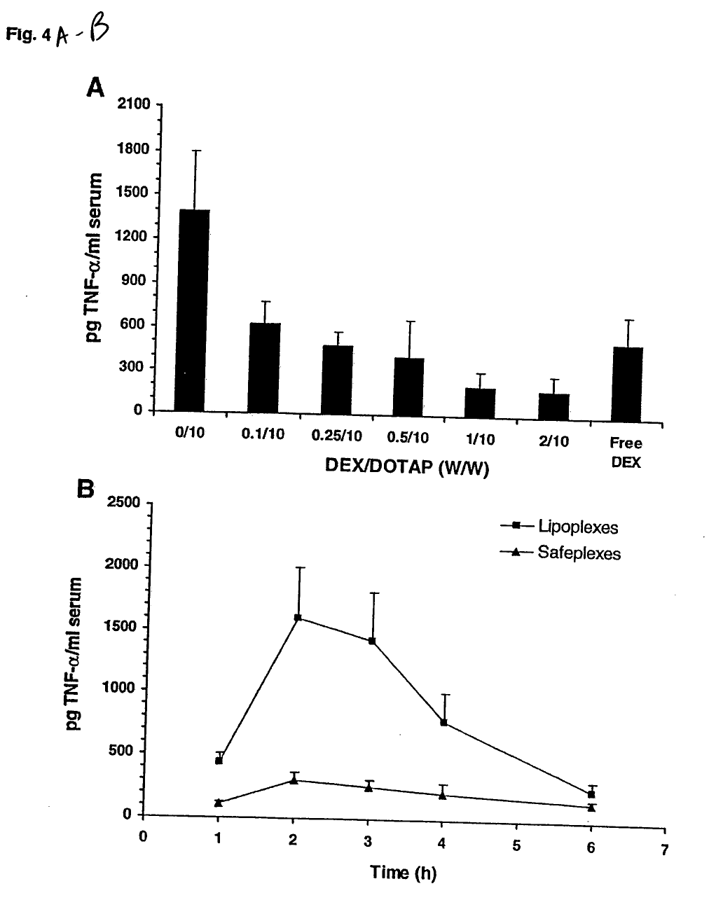

DEX dose effect experiment was designed to find out: 1) the minimal dose of DEX which could effectively inhibit TNF-α, and 2) the maximal amount of DEX which could be encapsulated into DOTAP liposomes. Each mouse was injected with the safeplexes containing 25 μg plasmid DNA, 32 μg DOTAP and varied amount of DEX (3.2, 8, 16, 32, and 64 μg). The safeplex, as shown in FIG. 4a, could inhibit approximately 50% of TNF-α production at a dose as low as 3.2 μg of DEX (DEX/DOTAP=0.1/10). The inhibition effect of DEX on TNF-α was dose dependent. As the dose of DEX increased to 32 μg (DEX/DOTAP=1/10, mol/mol=1/6) or higher (DEX/DOTAP=2/10, w/w), more than 80% inhibition of TNF-α was observed compared with less than 45% inhibition when 32 μg free DEX (Dexamethasone-Water Soluble, Sigma) was intravenously injected immediately followed by the injection of the DNA/DOTAP complexes. The co-delivered suppressor actually suppresses, and not simply delays TNF-α production, as evidenced by the time-course of TNF-α induction. The mice were injected with either the lipoplexes or safeplexes. The TNF-α, as shown in FIG. 4b, was induced as early as 1 h after the injection. The highest amount of TNF-α was observed 2 h after i.v. injection of lipoplexes and then decreased. The peak time for TNF-α production was similar to that reported previously (15). When mice were injected with the safeplexes, the pharmacokinetics profile in FIG. 4b indicated that the safeplexes suppressed TNF-α induction at all examined time points from 1 to 4 h (P<0.01). There was no significant difference in the levels of TNF-α for both lipoplexes and safeplexes 6 h after the injection, at which time the levels of TNF-α were low.

### EXAMPLE 7

### Effect of Other Candidate Suppressors on Inhibition of Cytokines

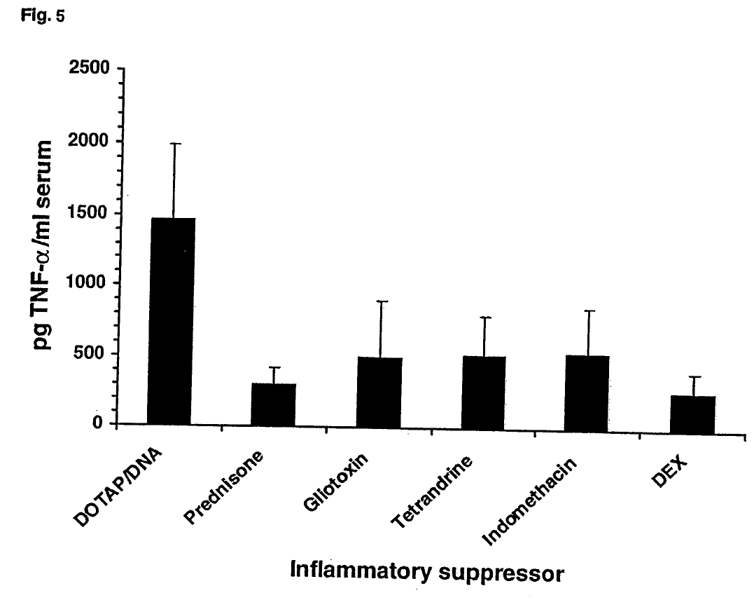

Glucocorticoids, for example DEX and prednisone, are widely used for their anti-inflammatory and immunosuppressive properties, which antagonize the activation of the NF-κB pathway by direct and indirect mechanisms (16). Other pharmacologic agents have been described to inhibit NF-κB activity and can be used in accordance with the present invention. These agents include non-steroidal anti-inflammatory drugs (NSAIDs) and natural compounds. Safeplex can carry, in addition to DEX, other inflammatory inhibitors such as prednisone (another glucocorticoid drug), indomethacin (NSAID), tetrandrine (herbal medicine) and gliotoxin (fungal metabolite). Gliotoxin, used as an inhibitor of NF-κB, exhibited profound immunosuppressive activity in vivo. Previous studies showed that sublethally irradiated mice with an intraperitoneal injection of gliotoxin showed a significant delay in the recovery of immune cells (17). Tetrandrine is a natural compound which was extracted from a Chinese herbal remedy known as Hanfngji (18). Tetrandrine has been used for immunosuppression (18), protective effects on hepatocyte injury, the treatment of ischemic heart diseases and anti-portal hypertension, and for anticarcinoma effects (19, 20). Tetrandrine has been shown to inhibit NF-κB activation via suppressing signal-induced degradation of IκBα (a cytoplasmic inhibitor of NF-κB transcription factor) (18). To test if other immune suppressors can be also encapsulated in cationic liposomes, the safeplexes containing the above selected pharmacologic agents were tested for the cytokine reduction. As shown in FIG. 5, all above inflammatory inhibitors showed the inhibitory effect on TNF-α production when they were carried by the safeplexes with a ratio of 1/6 (inhibitor/DOTAP, mol/mol). However, the NSAID and natural suppressors did not inhibit TNF-α production as efficiently as DEX.

### EXAMPLE 8

### Effect of Capsaicin and SB 203580 on Cytokine Inhibition

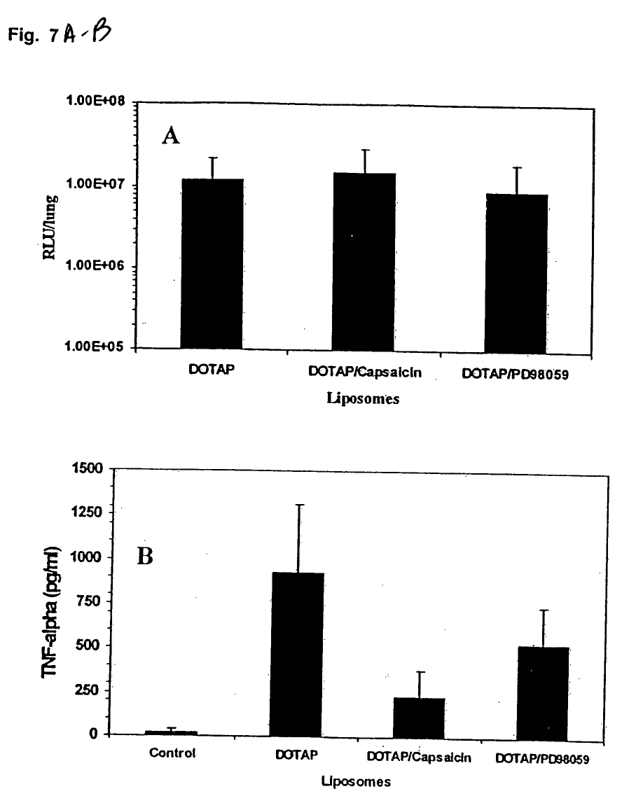

There are two major signaling pathways involved in cytokine production, which include, as shown in FIG. 6, the activation of NF-κB and AP-1 (30,31,32). We wanted to determine if inhibition of the NF-κB and AP-1 pathways with capsaicin and the commercially available SB 203580, respectively, would inhibit the production of the cytokine TNF-α. Like the safeplexes carrying DEX (FIG. 2b), safeplexes carrying capsaicin or SB 203580 transferred nucleic acid molecules to target cells as efficiently as DOTAP lipoplexes (FIG. 7a). Carrying capsaicin or SB 203580 did not affect the ability of safeplex vectors to transfer DNA to the target cells for transcription and translation.

Tumor necrosis factor a (TNF-α) is one of the major cytokines induced by CpG. It is known that a major organ involved in the production of cytokines is the lung (14). Theoretically, the inflammatory suppressors delivered by the safeplexes should accomplish their desired functions in this organ. When plasmid DNA was delivered by the safeplexes carrying capsaicin or SB 203580, the levels of TNF-α in the lung were significantly decreased when compared to the lipoplexes (FIG. 7b). The levels of TNF-α decreased 70% in response to the capsaicin safeplexes, and 40% in response to the SB 203580 safeplexes. These data indicate that the safeplex can serve as a non-immunostimulatory gene vector to inhibit the inflammatory toxicity induced by CpG motif pDNA.

### EXAMPLE 9

### Effect of FXR and CDCA Proteins on Nuclear Transcription of Safeplex Nucleic Acid Molecules

Currently, most efforts to design a new non-viral vector focus only on efficiently delivering DNA into cells, but lack additional functions such as the activation of nuclear transcription. Recent advances in molecular biology have greatly accelerated knowledge relating to nuclear transcription regulation. Therefore, a new generation of non-viral vectors should exhibit dual functions, delivering not only xenogenic transgenes but also some candidate ligands targeting nuclear receptor signalling, which can trigger the activation of transgene expression.

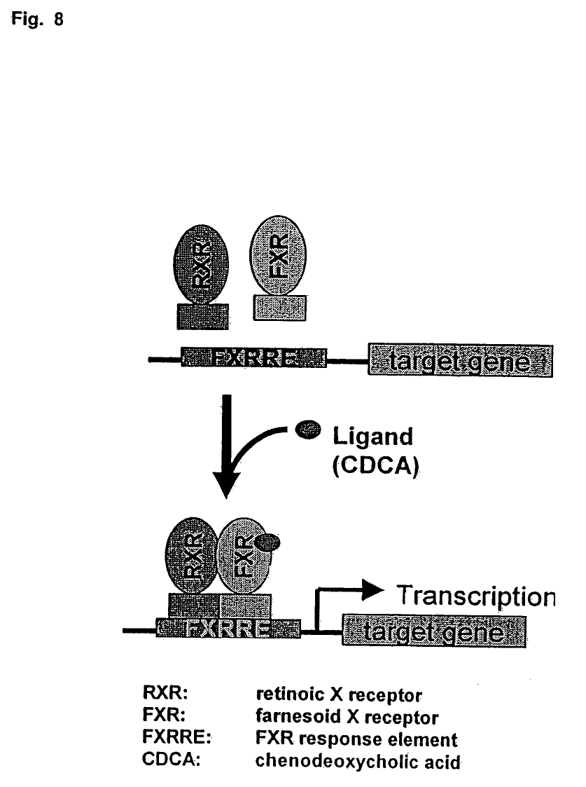

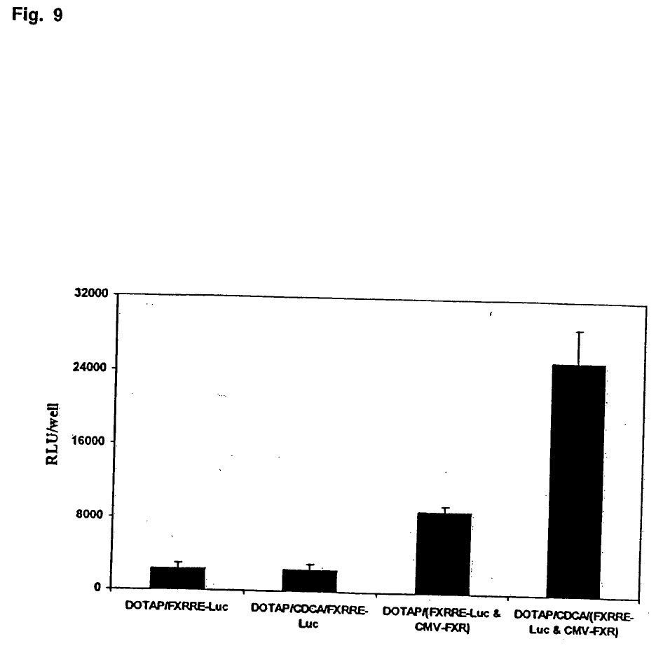

For this purpose, we used the safeplex vector to target the signalling pathway of the nuclear receptor FXR. FXR has been recently identified as a bile acid-activated nuclear receptor. FXR controls bile acid synthesis, conjugation, and transport, as well as lipid metabolism. In order to exhibit these functions, as shown in FIG. 8, FXR has to form a dimer with RXR (retinoic X receptor). The formation of the FXR/RXR dimer requires another small molecule, CDCA (chenodeoxycholic acid). Transcription will start when the dimer binds to the FXR response element (FXRRE) of a target gene. To test the idea of activating FXR-responsive genes with CDCA carried by the new vector, we incorporated CDCA into the DOTAP liposome, and then transferred DNA with DOTAP/CDCA into BL6 target cells. Very low levels of gene expression were detected for transfection of FXRRE-Luc (where luciferase was operably linked to the thymidine kinase promoter) with CDCA liposomes (FIG. 9). This is understandable because FXR mainly exists in the liver, kidney, and intestine not in this tumor cell line. When cells were transfected with two plasmids (one expressing the target luciferase gene and the other the FXR proteins) we observed some enhancement (FIG. 9). Further enhancement was observed when tranfecting using DOTAP/CDCA/FXRRE-Luc FXR liposomes (FIG. 9). The new non-viral safeplex vector not only efficiently delivers genes to target cells, but also inhibits proinflammatory cytokine production.

References

1. Niidome, T., and Huang, L. (2002) Gene therapy progress and prospects: nonviral vectors. Gene Ther 9, 1647-1652

2. Felgner, P. L., and Ringold, G. M. (1989) Cationic liposome-mediated transfection. Nature 337, 387-388

3. Liu, F., and Huang, L. (2002) Development of non-viral vectors for systemic gene delivery. J Control Release 78, 259-266

4. Audouy, S. A., de Leij, L. F., Hoekstra, D., and Molema, G. (2002) In vivo characteristics of cationic liposomes as delivery vectors for gene therapy. Pharm Res 19, 1599-1605

5. Scheule, R. K., St George, J. A., Bagley, R. G., Marshall, J., Kaplan, J. M., Akita, G. Y., Wang, K. X., Lee, E. R., Harris, D. J., Jiang, C., Yew, N. S., Smith, A. E., and Cheng, S. H. (1997) Basis of pulmonary toxicity associated with cationic lipid-mediated gene transfer to the mammalian lung. Hum Gene Ther 8, 689-707

6. Alton, E. W., Geddes, D. M., Gill, D. R., Higgins, C. F., Hyde, S. C., Innes, J. A., and Porteous, D. J. (1998) Towards gene therapy for cystic fibrosis: a clinical progress report. Gene Ther 5, 291-292

7. Freimark, B. D., Blezinger, H. P., Florack, V. J., Nordstrom, J. L., Long, S. D., Deshpande, D. S., Nochumson, S., and Petrak, K. L. (1998) Cationic lipids enhance cytokine and cell influx levels in the lung following administration of plasmid: cationic lipid complexes. J Immunol 160, 4580-4586

8. Tousignant, J. D., Gates, A. L., Ingram, L. A., Johnson, C. L., Nietupski, J. B., Cheng, S. H., Eastman, S. J., and Scheule, R. K. (2000) Comprehensive analysis of the acute toxicities induced by systemic administration of cationic lipid:plasmid DNA complexes in mice. Hum Gene Ther 11, 2493-2513

9. Li, S., Wu, S. P., Whitmore, M., Loeffert, E. J., Wang, L., Watkins, S. C., Pitt, B. R., and Huang, L. (1999) Effect of immune response on gene transfer to the lung via systemic administration of cationic lipidic vectors. Am J Physiol 276, L796-804

10. Yew, N. S., Wang, K. X., Przybylska, M., Bagley, R. G., Stedman, M., Marshall, J., Scheule, R. K., and Cheng, S. H. (1999) Contribution of plasmid DNA to inflammation in the lung after administration of cationic lipid:pDNA complexes. Hum Gene Ther 10, 223-234

11. Tan, Y., Li, S., Pitt, B. R., and Huang, L. (1999) The inhibitory role of CpG immunostimulatory motifs in cationic lipid vector-mediated transgene expression in vivo. Hum Gene Ther 10, 2153-2161

12. Krieg, A. M., Yi, A. K., Matson, S., Waldschmidt, T. J., Bishop, G. A., Teasdale, R., Koretzky, G. A., and Klinman, D. M. (1995) CpG motifs in bacterial DNA trigger direct B-cell activation. Nature 374, 546-549

13. Bird, A. P. (1986) CpG-rich islands and the function of DNA methylation. Nature 321, 209-213

14. Sakurai, F., Terada, T., Yasuda, K., Yamashita, F., Takakura, Y., and Hashida, M. (2002) The role of tissue macrophages in the induction of proinflammatory cytokine production following intravenous injection of lipoplexes. Gene Ther 9, 1120-1126

15. Tan, Y., Liu, F., Li, Z., Li, S., and Huang, L. (2001) Sequential injection of cationic liposome and plasmid DNA effectively transfects the lung with minimal inflammatory toxicity. Mol Ther 3, 673-682

16. Zingarelli, B., Sheehan, M., and Wong, H. R. (2003) Nuclear factor-kappaB as a therapeutic target in critical care medicine. Crit Care Med 31, S105-111

17. Pahl, H. L., Krauss, B., Schulze-Osthoff, K., Decker, T., Traenckner, E. B., Vogt, M., Myers, C., Parks, T., Warring, P., Muhlbacher, A., Czemilofsky, A. P., and Baeuerle, P. A. (1996) The immunosuppressive fungal metabolite gliotoxin specifically inhibits transcription factor NF-kappaB. J Exp Med 183, 1829-1840

18. Chen, F., Sun, S., Kuhn, D. C., Lu, Y., Gaydos, L. J., Shi, X., and Demers, L. M. (1997) Tetrandrine inhibits signal-induced NF-kappa B activation in rat alveolar macrophages. Biochem Biophys Res Commun 231, 99-102

19. Rao, M. R., Li, D. G., Wang, Z. R., and Lu, H. M. (2002) Effects of tetrandrine on cardiac and vascular remodeling

Pharmacology of tetrandrine and its therapeutic use in digestive diseases. Acta Pharmacol Sin 23, 1075-1085

20. Li, D. G., Wang, Z. R., and Lu, H. M. (2001) Pharmacology of tetrandrine and its therapeutic use in digestive diseases. World J Gastroenterol 7, 627-629

21. Yew, N. S., Zhao, H., Wu, I. H., Song, A., Tousignant, J. D., Przybylska, M., and Cheng, S. H. (2000) Reduced inflammatory response to plasmid DNA vectors by elimination and inhibition of immunostimulatory CpG motifs. Mol Ther 1, 255-262

22. Roman, M., Martin-Orozco, E., Goodman, J. S., Nguyen, M. D., Sato, Y., Ronaghy, A., Kornbluth, R. S., Richman, D. D., Carson, D. A., and Raz, E. (1997) Immunostimulatory DNA sequences function as T helper-1-promoting adjuvants. Nat Med 3, 849-854

23. Davis, H. L., Weeratna, R., Waldschmidt, T. J., Tygrett, L., Schorr, J., Krieg, A. M., and Weeranta, R. (1998) CpG DNA is a potent enhancer of specific immunity in mice immunized with recombinant hepatitis B surface antigen. J Immunol 160, 870-876

24. Gilkeson, G. S., Grudier, J. P., Karounos, D. G., and Pisetsky, D. S. (1989) Induction of anti-double stranded DNA antibodies in normal mice by immunization with bacterial DNA. J Immunol 142, 1482-1486

25. Niven, R., Pearlman, R., Wedeking, T., Mackeigan, J., Noker, P., Simpson-Herren, L., and Smith, J. G. (1998) Biodistribution of radiolabeled lipid-DNA complexes and DNA in mice. J Pharm Sci 87, 1292-1299.

26. Kobayashi, N., Kuramoto, T., Yamaoka, K., Hashida, M., and Takakura, Y. (2001) Hepatic uptake and gene expression mechanisms following intravenous administration of plasmid DNA by conventional and hydrodynamics-based procedures. J Pharmacol Exp Ther 297, 853-860.

27. Zuhom, I. S., Visser, W. H., Bakowsky, U., Engberts, J. B., and Hoekstra, D. (2002) Interference of serum with lipoplex-cell interaction: modulation of intracellular processing. Biochim Biophys Acta 1560, 25-36.

28. Hofman, C. R., Dileo, J. P., Li, Z., Li, S., and Huang, L. (2001) Efficient in vivo gene transfer by PCR amplified fragment with reduced inflammatory activity. Gene Ther 8, 71-74

29. Elouahabi, A., Flamand, V., Ozkan, S., Paulart, F., Vandenbranden, M., Goldman, M., and Ruysschaert, J. M. (2003) Free cationic liposomes inhibit the inflammatory response to cationic lipid-DNA complex injected intravenously and enhance its transfection efficiency. Mol Ther 7, 81-88

30. Hemmi, H., Takeuchi, O., Kawai, T., Kaisho, T., Sato, S., Sanjo, H., Matsumoto, M., Hoshino, K., Wagner, H., Takeda, K., and Akira, S. (2000) A Toll-like receptor recognizes bacterial DNA. Nature 408, 740-745

31. Mohan, R., and Heyman, R. A. (2003) Orphan nuclear receptor modulators. Curr Top Med Chem. 3, 1637-47

32. Jazirehi, A. R., and Bonavida, B. (2005) Cellular and molecular signal transduction pathways modulated by rituximab (rituxan, anti-CD20 mAb) in non-Hodgkin's lymphoma: implications in chemosensitization and therapeutic intervention. Oncogene. 24, 2121-43

33. Shockett, P. E. and Schatz, D. F. (1996) Diverse strategies for tetracycline=regulated inducible gene expression. Proc. Natl. Acad. Sci. U.S.A. 93, 5173-5176.

The present invention is not to be limited in scope by the specific embodiments described above. Many modifications of the present invention, in addition to those specifically recited above would be apparent to the skilled artisan using the teachings of the instant disclosure. Such modifications are intended to fall within the scope of the appended claims. All publications, patents and patent publications, cited above are herein incorporated by reference in their entireties.

We claim:

1. A liposomal vector comprising a cationic liposome, a nucleic acid molecule comprising a gene of interest in expressible form, and an inflammation suppressor.

2. The vector of claim 1 wherein the cationic liposome comprises 1,3-Dioleoyl-3-trimethylammonium-propane.

3. The vector of claim 1 wherein the nucleic acid molecule is a plasmid.

4. The vector of claim 1 wherein the nucleic acid molecule comprises a regulatory element.

5. The vector of claim 4 wherein the regulatory element is an enhancer.

6. The vector of claim 1 wherein the inflammation suppressor is selected from the group consisting of a glucocorticoid, a nonsteroidal anti-inflammatory drug, a NF-kappa B inhibitor, an AP-1 inhibitor, and a natural compound extracted from an herb.

7. The vector of claim 6 wherein the glucocorticoid is selected from the group consisting of betamethasone, budesonide, cortisone, dexamethasone, hydrocortisone, methylprednisolone, prednisolone, prednisone, and triamcinolone.

8. The vector of claim 6 wherein the nonsteroidal anti-inflammatory drug is selected from the group consisting of indomethacin, ibuprofen, naproxen, aspirin, acetaminophen, and an agent that inhibits cyclooxygenase.

9. The vector of claim 6 wherein the natural compound is selected from the group consisting of tetrandrine, extract of Periwinkle, and extract of Tripterygium.

10. The vector of claim 6 wherein the NF-kappa B inhibitor is gliotoxin or capsaicin.

11. The vector of claim 6 wherein the AP-1 inhibitor is SB 203580.

12. A method of delivering a nucleic acid molecule in combination with an inflammation suppressor to a cell, the method comprising administering to the cell a vector comprising a cationic liposome, a nucleic acid molecule comprising a gene of interest in expressible form, and an inflammation suppressor(s), wherein production of a cytokine by the cell in response to the nucleic acid molecule is inhibited.

13. The method of claim 12 wherein the cytokine is selected from the group consisting of neurotrophin, tumor necrosis factor, interleukin, transforming growth factor, and fibroblast growth factor.

14. A method of delivering a nucleic acid molecule in combination with an inflammatory suppressor to a cell in a subject comprising administering to the subject an effective amount of a vector comprising a cationic liposome, a nucleic acid molecule comprising a gene of interest in expressible form, and an inflammation suppressor, wherein production of a cytokine by the cell in response to the nucleic acid molecule is inhibited.

15. The method of claim 14 wherein the cytokine is selected from the group consisting of neurotrophin, tumor necrosis factor, interleukin, transforming growth factor, and fibroblast growth factor.

16. The method of claim 14 wherein the administering is by intravenous injection or intraperitoneal injection.

17. A method of identifying an anti-inflammatory agent, the method comprising contacting a vector comprising a cationic liposome, a nucleic acid molecule, and the agent with a cell, and measuring the immune response to the vector relative to a control vector.

18. The method of claim 17 wherein the agent is obtained from a laboratory chemical synthesis.

19. The method of claim 17 wherein the agent is obtained from an herb.

20. A liposomal vector comprising a cationic liposome, a nucleic acid molecule comprising a gene of interest in expressible form, and a component that enhances expression of the gene of interest.

21. The vector of claim 20, wherein the component provides a transcriptional regulator that binds to a transcriptional control region operably linked to the gene of interest.

22. The vector of claim 21, wherein the component is a nucleic acid encoding the transcriptional regulator.

23. The vector of claim 21, wherein the component is a transcriptional regulator protein.

24. The vector of claim 20, wherein the component is a ligand that binds to a transcriptional regular protein.

25. The vector of claim 20, wherein the component is a nucleic acid encoding the farnesoid X receptor.

26. The vector of claim 20, wherein the component is chenodeoxycholic acid.

27. The vector of claim 20, further comprising an inflammation suppressor.

28. The vector of claim 27, wherein the inflammation suppressor is selected from the group consisting of a glucocorticoid, nonsteroidal anti-inflammatory drug, NF-kappa B inhibitor, AP-1 inhibitor, a natural compound extracted from an herb, betamethasone, budesonide, cortisone, dexamethasone, hydrocortisone, methylprednisolone, prednisolone, prednisone, triamcinolone, indomethacin, ibuprofen, naproxen, aspirin, acetaminophen, and an agent that inhibits cyclooxygenase, tetrandrine, extract of Periwinkle, extract of Tripterygium, gliotoxin, capsaicin, and SB 203580.

29. A method of delivering a gene of interest to a cell, comprising contacting the cell with an effective amount of a vector according to claim 20.

30. A method of delivering a gene of interest to a cell, comprising contacting the cell with an effective amount of a vector according to claim 27.

31. A method of delivering a gene of interest to a cell, comprising contacting the cell with an effective amount of a vector according to claim 28.

32. The method of claim 29, where the cell is in a subject.

33. The method of claim 30, where the cell is in a subject.

34. The method of claim 31, where the cell is in a subject.
